SEQUENCE LISTING (1 sequence)

SEQ ID NO: 1 - protein, 426 aa, Chicken
  Chain (1)..(123)
  ProPeptide (124)..(369)
  ref <308>: DATABASE ACCESSION NUMBER: Uniprot/P02460
  ref <309>: DATABASE ENTRY DATE: 1986-07-21
  ref <313>: RELEVANT RESIDUES IN SEQ ID NO: (1)..(369)
SPPCACHICKCLLAGENALPHAIICHAINFRAGMENTSGALLSGALLSGNCLAPESVGETGEAGERGLKGHRGFTGLQGLPGPPGPSGDQGAAGPAGPSGPRGPPGPVGPSGKDGSNGMPGPIGPPGPRGRSGEPGPAGPPGNPGPPGPPGPPGTGIDMSAFAGLGQTEKGPDPIRYMRADEAAGGLRQHDVEVDATLKSLNNQIESIRSPEGSKKNPARTCRDIKLCHPEWKSGDYWIDPNQGCTLDAIKVFCNMETGETCVYPTPSSIPRKNWWTSKTKDKKHVWFAETINGGFHFSYGDENLSPNTASIQMTFLRLLSTEGSQNVTYHCKNSIAYMDEETGNLKKAILIQGSNDVEIRAEGNSRFTYSVLEDGCTKHTGKWGKTVIEYRSQKTSRLPIVDIAPMDIGGADQEFGVDIGPVCFL

(19) United States
(12) Patent Application Publication
(10) Pub. No.: US 2020/0376090 A1
(43) Pub. Date: Dec. 3, 2020

(54) COMBINED COLLAGEN NSAID ANALGESIC DOSAGE REGIMEN

(71) Applicant: Performance By Science, LLC, Lafeyette, CA (US)

(72) Inventor: Mark Terrell Smith, Lafayette, CA (US)

(73) Assignee: Performacne By Science, LLC, Lafayette, CA (US)

(21) Appl. No.: 16/425,456

(22) Filed: May 29, 2019

(51) Int. Cl. A61K 38/39; A61K 31/616; A61K 31/192; A61K 31/415; A61K 31/196; A61K 31/407; A61K 31/405; A61K 31/12; A61K 31/421; A61K 31/5415; A61K 31/618; A61K 9/00

(52) U.S. Cl. CPC A61K 38/39; A61K 31/616; A61K 31/192; A61K 31/415; A61K 31/196; A61K 9/0053; A61K 31/405; A61K 31/12; A61K 31/421; A61K 31/5415; A61K 31/618; A61K 31/407

(57) ABSTRACT

This invention generally relates to pharmaceutical compositions for use in the treatment of pain and inflammation, the prevention and improvement of symptoms of musculoskeletal distress degeneration, including temporary loss of range of motion, temporary inflammation, temporary muscle soreness, and combinations thereof, to assist recovery following exercise and recovery of muscles and joints therefrom, and to provide relief of such symptoms in a mammalian organism, said compositions comprising: (i) an analgesically and anti-inflammatory effective amount of a undenatured cartilage including an isolated native Type II collagen; and (ii) an analgesically and antitussively effective amount of at least one non-steroidal anti-inflammatory agent including aspirin, celecoxib, diclofenac, ibuprofen, indomethacin, naproxen, oxaprozin and piroxicam, or a therapeutically active stereoisomer thereof substantially free of its other stereoisomers; and optionally (iii) a pharmaceutically acceptable amount of at least one excipient.

### RELATED APPLICATIONS

This utility application claims the benefit of the priority of the following United States patent applications, U.S. Provisional application Ser. No. 62/101,893, filed on Jan. 9, 2015, U.S. Utility Patent application Ser. No. 14/990,248, filed on Jan. 7, 2016, and U.S. Provisional application Ser. No. 62/682,043, filed on Jun. 7, 2018, all of which are incorporated in their entirety by reference herein.

### FIELD OF THE INVENTION

This invention generally relates to pharmaceutical compositions for use in the treatment of pain and inflammation, the prevention and improvement of symptoms of arthritis and musculoskeletal distress degeneration, including loss of range of motion, inflammation, muscle soreness, and combinations thereof, to assist recovery following exercise and recovery of muscles and joints therefrom, and to provide relief of such symptoms in a mammalian organism, wherein said compositions are a combination of an analgesically and anti-inflammatory effective amount of an undenatured or native cartilage in the form of an isolated native collagen, such as a native Type II collagen; and an analgesically and antitussively effective amount of at least one analgesic compound selected from a non-steroidal anti-inflammatory agent (NSAID), optionally an opioid analgesic, and optionally a therapeutically effective amount of at least one excipient, being a pharmaceutically acceptable carrier, filler material or adjuvant.

### BACKGROUND

The present invention relates generally to the treatment of symptoms related to musculoskeletal distress, commonly treated using anti-inflammatory drugs, most preferably the subgroup of compounds known as non-steroidal anti-inflammatory drugs (NSAID). These materials have been utilized in the treatment of pain and inflammation and have been disclosed as useful in the treatment, management and mitigation of muscle aches, fatigue, soreness and pain associated with exercise as well as a trauma resulting from such related activities, work and sports. Further, NSAID have been found to be effective in the treatment of more severe medical conditions, including arthritis (both osteoarthritis and rheumatoid arthritis) and osteopenia, where the action of the NSAID have been found to include antipyretic action. Not only pain accompanying arthrosis can be reduced, but the mobility of the joints can be increased. In the case of chronic rheumatoid arthritis, ibuprofen, a common over-the-counter (OTC) NSAID, has been found to reduce pain, joint swelling and morning stiffness, and to improve the functionality of the joints in mammals. Owing to the constant wear and tear and subsequent metabolic response to restore cartilage associated with joints, connective tissue and the body's newly documented collagen-rich interstitium regions, collagen supplements are taken to provide the body with an excess of available collagen for the building and maintenance of healthy joints.

In addition, muscles, joints and tendons associated with the musculoskeletal system are subject to normal wear and tear during regular day to day activity and additional stress and damage during exercise and work, benefiting from dietary supplementation of collagen, preferentially collagen derived from cartilage, and even more preferentially native collagens. Further, native Type II collagen derived from avian and mammalian sources is highly beneficial in supplying collagen in a readily assimilable and human compatible form. Dietary supplementation with suitable collagen sources are therefore highly desirable, and may further be extremely beneficial in reducing or treating existing symptoms or preventing the user from incurring symptoms resulting from musculoskeletal distress such as a measurable decrease in the range of movement, a measurable increase in muscle soreness, or a measurable amount of inflammation resulting from physical exertion, and/or to reduce or prevent longer-term detrimental effects associated with such physical activities, such as bone, cartilage or ligament wear, thinning or damage, and the like.

Accordingly, the present invention further relates to a method to effectively combine the use of native collagen with an NSAID in order to both treat the symptoms of musculoskeletal distress and to provide materials needed to repair damage to cartilage and generate new cartilage. Generally, inflammation and swelling, and the pain associated with therewith as well as tissue and musculoskeletal stress or damage, is initially higher just after exercise or injury, triggered in part by the human body's immune system reaction to injury, which includes, but may not be limited to, increased initial blood flow and eventually some degree of inflammation, swelling or vascular constriction at and around the site of injury. Thus, some pain medication may be taken initially to treat the immediate symptoms of discomfort and pain, and then stopped when the threshold of pain drops to an acceptable level, which may and does differ between individuals, as well as depending on other factors.

Further, in the instances of more severe injury or pain, the NSAID analgesic may optionally be augmented by the use of a stronger analgesic agent, such as for example, an opioid analgesic.

Further, to assist in the formulation, preparation and formation of tablets, capsules and other dosing media for compositions of the present invention, the embodiments may also include an excipient and/or filler material to provide at least one additional benefit or feature, or to provide bulk, filling, ease of handling and/or identification means to the final formulated product form, respectively.

Individuals who, due to a variety of factors including age, injury, repetitive stress, regular exercise, physical activity or bearing heavy loads, frequently experience short term side effects that include temporary loss of range of motion, inflammation, or muscle soreness. These conditions, which may lead to long term effects such as bone wear, weakened tendons or other chronic issues. In most cases, such symptoms do not require clinical treatment and a medical prescription is not necessary, however, they do interfere with regular activities such as work, exercise and child-care. Existing commercial oral remedies often require multi-gram doses, and have low compliance and/or low response rates. It would be desirable to have a composition that can be delivered in a small and effective daily dose, preferably less than 1 gram per serving per day, and even more preferably less than 100 milligrams per day, which/that can reduce or prevent or remove both measurable short term symptoms of distress and/or chronic long-term and measurable health symptoms associated with musculoskeletal wear and metabolic imbalances resulting from high physical activity levels, physical exertion, sports, or repetitive motions.

It would be desirable to have a composition that uses safe and effective ingredients that do not require oversight by a physician. Still further, it would be desirable to have a composition that may be administered orally without sophisticated medical technology, and would be effective by more than one mechanism of action so as to increase the percentage of the population for which it is effective. Therefore, there currently exists a need in the industry for a composition and/or an associated method to counteract symptoms of musculoskeletal distress in the broad population and slow degenerative processes by administering a small effective dose of a composition comprised by a collagen, and at least one acceptable non-steroidal anti-inflammatory agent (NSAID), optionally in a controlled dosage regimen where the synergistic effects of the two treatment aids can be used advantageously to treat the pain and symptoms, resulting in reduced dependency on pain medications.

Further, it would also be desirable to have a programmed set of compositions that may be easily administered or taken by a patient, wherein the compositions provide a constant dosage of one selected medicant and one variable dosage of a second selected analgesic agent over a preselected treatment period, for convenience of use and for the purpose of slowly reducing the need for and exposure to the patient to the analgesic agent as symptoms of discomfort, inflammation, pain, soreness, swelling and the like diminish over time during the course of treatment over that preselected treatment period.

Furthermore, it would also be desirable to have a personalized programmed set of compositions that may be easily administered or taken by a patient, wherein the compositions provide a personally selected or tailored yet constant dosage of one selected medicant and one personally selected or tailored yet variable dosage of a second selected analgesic agent over a preselected treatment period, for convenience of use and for the purpose of slowly reducing the need for and exposure to the patient to the analgesic agent as symptoms of discomfort, inflammation, pain, soreness, swelling and the like diminish over time during the course of treatment over that preselected treatment period, but tailored to the individual person or patient in view of the degree or magnitude of the initial injury and symptoms to be treated.

### SUMMARY

One object of the present invention are compositions and a method to treat and alleviate symptoms of musculoskeletal inflammation by administering a dietary oral dosage regimen comprising a therapeutic amount of a native or undenatured, biologically active collagen; a therapeutic amount of an analgesic compound comprising a non-steroidal anti-inflammatory drug selected from aspirin, ibuprofen, and combinations thereof; and optionally, a pharmaceutically acceptable excipient; wherein said dietary dosage regimen comprises a treatment period whose duration is selected from 1 week to 1 year.

Another object of the present invention are compositions and methods wherein said native collagen is a native Type II collagen.

A further object of the present invention are compositions and methods wherein said native Type II collagen is derived from an avian source.

Yet a further object of the present invention are compositions and methods wherein said avian source is chicken and cartilaginous components thereof.

Another object of the present invention are compositions and methods wherein said therapeutic amount of said analgesic compound varies over said treatment period; wherein said amount of analgesic is provided at a first dosage level during a first series of administrations; followed by a second dosage level over a second series of administrations; and optionally followed by an n-plurality of additional dosage levels over said plurality of series of continuing administrations; wherein said dosage regimen delivers a total of n+2 administration series over said duration of said treatment period.

Yet another object of the present invention are compositions and methods is use of a collagen wherein said native Type II collagen corresponds to an expressed cross-linked polypeptide coded by the nucleic acid molecule consisting of a sequence of bases encoding the amino acid sequence of Seq. ID. No. 1, as identified hereinbelow and in FIG. 4.

A further object of the present invention are compositions and methods wherein each of said second and n-plurality of additional dosage levels deliver dosage levels of said analgesic at a lower level than said first dosage level.

Yet a further object of the present invention are compositions and methods wherein each of said n-plurality of additional dosage levels deliver dosage levels of said analgesic at progressively lower levels than said first and said second dosage levels.

Another object of the present invention are compositions and methods wherein said final n+2 dosage levels of said analgesic compound corresponds to a subtherapeutic dosage level of said analgesic, or has no level of said analgesic at all.

Yet another object of the present invention are compositions and methods wherein said total of administration series corresponds to an oral dosage regiment extending over a time period selected from 2, 4, 6, 8 and 12 weeks.

One other object of the present invention are compositions and methods for the inhibition of an arthritis disease process in a patient in need thereof, the method comprising: orally administering a non-steroidal anti-inflammatory drug (NSAID) or pharmaceutically acceptable salts thereof; and a native Type II collagen in synergistic amounts and in a ratio of NSAID:collagen from about 80:1 to about 12,000:1; and for a duration effective to inhibit an arthritis disease process in a patient in need thereof.

A further object of the present invention are compositions and methods wherein said non-steroidal anti-inflammatory drug (NSAID) is selected from aspirin, aspirin salsalate, celecoxib, diclofenac, diflunisal, etodolac, ibuprofen, indomethacin, ketoprofen, ketorolac, nabumetone, naproxen, oxaprozin, piroxicam, salsalate, sulindac, and tolmetin.

Yet a further object of the present invention are compositions and methods wherein at least one of said NSAIDs are in an oral dosage form suitable for dosing once or twice a day over a treatment period.

And yet another object of the present invention are compositions and methods wherein said NSAID is administered in a daily dose ranging from about 100 to about 2,400 milligrams and said collagen is administered in a daily dose ranging from about 50 to about 500 micrograms.

One further object of the present invention are compositions and methods wherein said arthritis disease is selected from osteoarthritis, rheumatoid arthritis or osteopenia, or combinations thereof.

Yet another object of the present invention are compositions and methods consistent with the above objects of invention wherein said native collagen is a native Type II collagen, or alternatively, a native Type II collagen corresponding to an expressed cross-linked polypeptide coded by the nucleic acid molecule consisting of a sequence of bases encoding the amino acid sequence of Seq. ID. No. 1, as shown below and illustrated in FIG. 4.

One more object of the present invention are compositions and methods wherein said oral dosage form comprises a variable amount of 5 to 250 milligrams of a NSAID administered over said treatment period and a fixed amount of 50 to 500 micrograms of a native collagen administered over said treatment period.

Additionally, an object of the present invention are compositions and methods wherein said dosage form comprises a first dosage level during a first series of administrations; followed by a second dosage level over a second series of administrations; and optionally followed by an n-plurality of additional dosage levels over said plurality of series of continuing administrations; wherein said dosage regimen delivers a total of n+2 administration series over said duration of said treatment period, wherein said treatment period can begin again for an additional period of time.

A final object of the present invention are compositions and methods wherein said NSAID and said collagen are present in synergistic amounts over the duration of said treatment period and present in a synergistic ratio of NSAID:collagen from about 80:1 to about 12,000:1; wherein said synergistic ratio varies over said treatment period; and wherein said treatment period is of a duration effective to inhibit an arthritis disease process in a patient in need thereof, and wherein an opioid may also be formulated in addition to the NSAID in a similar manner to provide a programmed dosage of a combined analgesic combination and a collagen for the purposes of treating a mammal suffering musculoskeletal distress disorders including arthritis, osteoarthritis, osteopenia and the like.

### BRIEF DESCRIPTION OF THE DRAWINGS

FIG. 1 shows a composition chart of a 10 week series of treatment compositions for a variable dosage form of the present invention.

FIG. 2 shows a formulation-style chart of the 10 week series of treatment compositions for a constant weight dosage form of the present invention.

FIG. 3 shows one embodiment of a blister pack dispensing means for delivery a four week treatment regime.

FIG. 4 shows the sequence coding of a collagen coded by the COL2A1 gene, as derived from avian and mammalian sources.

### DESCRIPTION

### Generality of Invention

This application should be read in the most general possible form. This includes, without limitation, the following:

References to specific techniques include alternative and more general techniques, especially when discussing aspects of the invention, or how the invention might be made or used.

References to “preferred” techniques generally mean that the inventor contemplates using those techniques, and thinks they are best for the intended application. This does not exclude other techniques for the invention, and does not mean that those techniques are necessarily essential or would be preferred in all circumstances.

References to contemplated causes and effects for some implementations do not preclude other causes or effects that might occur in other implementations.

References to reasons for using particular techniques do not preclude other reasons or techniques, even if completely contrary, where circumstances would indicate that the stated reasons or techniques are not as applicable.

Furthermore, the invention is in no way limited to the specifics of any particular embodiments and examples disclosed herein. Many other variations are possible which remain within the content, scope and spirit of the invention, and these variations would become clear to those skilled in the art after perusal of this application.

Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

Read this application with the following terms and phrases in their most general form. The general meaning of each of these terms or phrases is illustrative, not in any way limiting.

### DETAILED DESCRIPTION

In general, the treatment of arthritic conditions has been limited to symptomatic treatment, for example to relieve symptoms such as inflammation and pain. Thus, for example, it has been proposed to use so-called non-steroidal anti-inflammatory drug (NSAID's) in the treatment of arthritic conditions. It has also been proposed to use a variety of analgesics, including opioid analgesics, in the relief of pain in arthritic conditions. It has now been found, in accordance with the present invention, that the treatment of arthritic conditions with both (i) an NSAID, and (ii) a source of collagen can serve to treat the arthritic condition itself, that is to inhibit the arthritic process and associated symptomology, and further supply collagen necessary for the repair, rebuilding and replenishment of cartilaginous elements of the musculoskeletal system. Accordingly, one embodiment of the present invention provides the use of an NSAID together with a source of collagen in the manufacture or preparation of a medicament for the treatment of arthritis (both osteoarthritis and rheumatoid arthritis) and osteopenia, and the symptoms associated therewith.

The present invention generally relates to a method of treating musculoskeletal distress in healthy active humans and animals comprising administering a native collagen, such for example a native Type II collagen in an effective amount sufficient to reduce at least one symptom resulting from said musculoskeletal distress; wherein said symptom is selected from temporary loss of range of motion, temporary inflammation, temporary muscle soreness, and combinations thereof, while also simultaneously administering an analgesic drug in a second effective amount sufficient to address the symptoms of said distress, i.e., discomfort, inflammation, swelling and pain associated therewith.

The present invention further relates generally to a method of treating the symptoms of osteoarthritis (OA), rheumatoid arthritis (RA), osteopenia and joint pain caused by repetitive exercise/motion and musculoskeletal distress, while simultaneously providing one additional health benefit comprising administering (a) a native collagen, such as for example a native Type II collagen; and (b) at least one non-steroidal anti-inflammatory agent, or a therapeutically active stereoisomer thereof substantially free of its other stereoisomers and (c) optionally, a therapeutically effective amount of at least one excipient, or a pharmaceutically acceptable salt thereof.

### Collagen

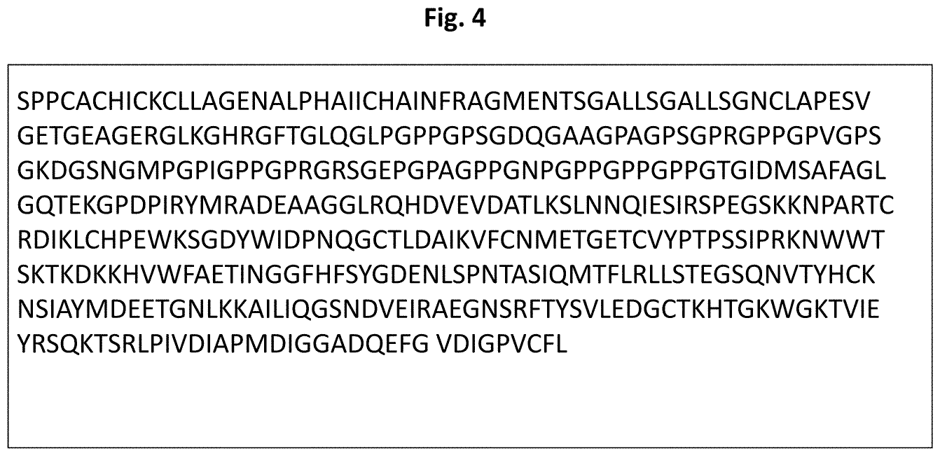

In the embodiments of the present invention, an acceptable collagen includes a native collagen. In an alternative embodiment, the collagen is preferably derived from an avian or mammalian source. In another embodiment of the present invention, the collagen is selected from a native Type II collagen source that has not been enzymatically processed or hydrolyzed in order to maintain the integrity of the native state of the collagen, i.e. prevent it from denaturing during harvesting, isolation, preparation and formulation into a dietary supplement form. In another embodiment, the acceptable type of collagen Type II is a collagen source expressed via the COL2A1 gene and identified as having a greater than a 98% match of the following amino acid sequence according to a BLAST protein identification search, also shown in FIG. 4 and identified herein as Seq. ID. No. 1:

SPPCACHICKCLLAGENALPHAIICHAINFRAGMENTSGALLSGALLSGN
CLAPESVGETGEAGERGLKGHRGFTGLQGLPGPPGPSGDQGAAGPAGPSG
PRGPPGPVGPSGKDGSNGMPGPIGPPGPRGRSGEPGPAGPPGNPGPPGPP
GPPGTGIDMSAFAGLGQTEKGPDPIRYMRADEAAGGLRQHDVEVDATLKS
LNNQIESIRSPEGSKKNPARTCRDIKLCHPEWKSGDYWIDPNQGCTLDAI
KVFCNMETGETCVYPTPSSIPRKNWWTSKTKDKKHVWFAETINGGFHFSY
GDENLSPNTASIQMTFLRLLSTEGSQNVTYHCKNSIAYMDEETGNLKKAI
LIQGSNDVEIRAEGNSRFTYSVLEDGCTKHTGKWGKTVIEYRSQKTSRLP
IVDIAPMDIGGADQEFGVDIGPVCFL

The COL2A1 label is the scientific descriptor for the gene responsible for the production of the alpha1(II) chain of type II collagen, the COL2A1 gene being located on the long (q) arm of chromosome 12 in region 12q13.11-13.2 of the human genome and mammalian equivalents. Mutations in the COL2A1 gene lead to a number of different heritable skeletal disorders, including achondrogenesis type II, hypochondrogenesis, Kniest dysplasia, spondyloepimetaphyseal dysplasia of the Strudwick type, spondyloepiphyseal dysplasia congenita, and Stickler syndrome.

Type II collagen, which adds structure and strength to connective tissues, is found primarily in cartilage, the gel that fills the eyeball (vitreous body), inner ear, and the center portion of the discs between the vertebrae in the spine (nucleus pulposus).

There are two forms of type II collagen made in the body. One version, type IIA, is made mainly in the vitreous body of the eye. The second version, type IIB, is preferentially produced and present in adult cartilage tissue.

Collagen II is initially produced as Type II procollagen, a protein consisting of three pro-alpha1(II) chains twisted together to form a triple-stranded helical (spiral-shaped) molecule. While in the cell, enzymes modify certain amino acids (the building blocks of proteins), specifically lysine and proline, by adding chemical groups that are necessary for the strands to come together in a stable structure and then cross-link with other molecules. Other enzymes add sugars to the protein. The triple-stranded type II procollagen molecule leaves the cell and is converted to collagen by enzymes that cleave small segments off both ends. The collagen molecules arrange themselves into long, thin fibrils outside of the cell. The fibrils come together in side-by-side groups to form collagen fibers. Cross-linking between molecules in fibrils produces a very stable protein structure, which contributes to collagen's tissue-strengthening function.

The dosage of collagen may vary depending on the nature of the musculoskeletal disease, damage or injury to be treated, the patient's sex, age, physical conditioning, body mass and the extent of the inflammation, injury, pain and swelling and other symptoms sought to be treated. In embodiments of the present invention, the daily dosage of collagen can vary between 50 micrograms (mcg) to 10.0 gram (gm), or alternatively between 0.1 milligrams (mg) to 5 gm, or yet alternatively between 0.25 mg to 1,000 mg, or yet alternatively between 0.5 mg to 500 mg, either as a total dosage or as a relative dosage per 100 kilograms (kg) of the subject weight or body mass. As collagens are a form of natural protein, it is very unlikely to have an upper bound or toxicity level that would limit the daily acceptable ingestion limit, or exhibit undesirable side-effects or adverse symptomology when taken even at a level of a magnitude higher in dosage than the typical daily dosages disclosed herein.

Accordingly, undenatured or native forms of the collagen Type II are preferentially selected for the purposes of the instant invention, being in a stable and biologically accessible and active form that is capable of promoting or invoking an immunological response while present in the body, primarily within the gastro-intestinal system. To invoke the desired immune response, the collagen is preferably in a chemical state with at least it's secondary, two dimensional structure (amino acid sequence) intact, or even more preferably in a chemical and physical conformational state with its tertiary, three-dimensional structure (folded, biologically active or “native” state) intact, or alternatively in an associated quaternary conformational state present as a fibrillar assembly of collagen fibers (cellular structural state). Type II collagens, and particularly those inventive variants disclosed herein are particularly stable and biologically active, producing a high degree of oral tolerance when administered through ingestion, particularly when in an undenatured state, and most particularly in its biologically active, native state. (See “Collagen Structure And Stability”, Annu Rev Biochem. 2009 ; 78: 929-958).

Oral tolerance translates to a diminished immune response to other allergens and antigens, and the patient's own collagen as a result of a prior exposure to an immunological response-invoking agent, such as the inventive collagens. Repeated oral administrations of Type II collagen (CII) induces oral tolerance. Without being bound by theory, it is believed that mammalian dendritic cells and subsets (DCs) in the gut or intestinal track become associated with lymphoid tissue (GALT) which acts to take up the collagen in whole (its native state) and then present it to T cells, which in turn respond to the exposure by generating regulatory T cells (T_{regs}), which induce systemic immune tolerance to Type II collagen. Inhibitory cytokines, such as transforming growth factors (TGF-beta), interleukin (IL)-10, and several other immune regulatory molecules, including indoleamine 2,3-dioxygenase (IDO) and retinoic acid, play an important role in T_{reg} generation. Each DC subset may play different roles, and integrin glycoproteins expressed by dendritic cells (such as CD11c and CD11b) interact with a small subset of lymphocytes generating either DCs and IDOs, which result in the favorable generation of antigen-inducible T_{reg}'s following CII oral tolerance. Upon stimulation with the antigen involved in its generation, T_{reg}'s are activated and regulate the immune response through inhibitory cytokine production, cell-to-cell contact-dependent mechanisms, DC modification, and bystander suppression. The DCs and T_{reg}'s are thus deeply involved in oral tolerance through reciprocal interactions. Accordingly, the native or fully undenatured biologically active three-dimensional form of Type II collagen is critical to induce oral tolerance and the resulting cascade of immune response mechanisms in the mammalian body that serve to protect and stimulate the repair and growth of new collagen where immune response, damage or stress to an existing collagen network has been triggered.

Prostaglandins are a family of chemicals that are produced by the cells of the body and have several important functions. They promote inflammation that is necessary for healing, but also results in pain, and fever; support the blood clotting function of platelets; and protect the lining of the stomach from the damaging effects of acid. Prostaglandins are produced within the body's cells by the enzyme cyclooxygenase (COX). There are two COX enzymes, COX-1 and COX-2. Both enzymes produce prostaglandins that promote inflammation, pain, and fever. However, only COX-1 produces prostaglandins that support platelets and protect the stomach. Nonsteroidal anti-inflammatory drugs (NSAIDs) block the COX enzymes and reduce prostaglandins throughout the body. As a consequence, ongoing inflammation, pain, and fever are reduced. Since the prostaglandins that protect the stomach and support platelets and blood clotting also are reduced, some NSAIDs can cause ulcers in the stomach and promote bleeding, depending in part on dosage present as well as individual sensitivity. Accordingly, in one embodiment of the instant invention, the NSAID are formulated with an enteric coating that delays or prevents the release of the drug within the stomach, but dissolves away to effectively release the drug once it has left the stomach and entered the intestinal track for absorption into the body.

Representative NSAIDs suitable for use with the instant invention include, but are not limited to, aspirin, aspirin salsalate (Amigesic™), celecoxib (Celebrex™), diclofenac (Cambia™, Cataflam™, Voltaren-XR™, Zipsor™, Zorvolex™), diflunisal (Dolobid—discontinued brand), etodolac (Lodine—discontinued brand), ibuprofen (Motrin™, Advil™), indomethacin (Indocin™), ketoprofen, ketorolac (Toradol—discontinued brand), nabumetone (Relafen—discontinued brand), naproxen (Aleve™, Anaprox™, Naprelan™, Naprosyn™), oxaprozin (Daypro™), piroxicam (Feldene™), salsalate (Disalsate™), sulindac (Clinoril—discontinued brand) and tolmetin (Tolectin—discontinued brand).

Without being bound by theory, it is believed that the combination of an anti-inflammatory drug and a collagen provides a synergistic benefit by reducing inflammation at the site of damage or an injury, temporarily reducing the body's defensive mechanisms and enabling an increased flow of blood into the compromised site and the decreased retention of fluids and lymphatic secretions in and around the site of damage or injury, thus enabling the localized absorption and utilization of the supplied collagen to aid in tissue and body collagen repair and replacement otherwise impaired by swelling and edema associated with the body's immune response mechanism. Further, by taking an analgesic that helps to reduce pain, the person suffering from musculoskeletal distress or an arthritic condition will be more inclined to move the affected joints and associated musculature, which is beneficial in maintaining blood flow to the region as well as the specific site of injury or damage, which helps reduce the symptoms and supports faster repair and recovery simply by easing pain.

The following Table 1 presents a list of common NSAIDs that may be suitable employed in the compositions and methodologies of the present invention, showing the daily dosage ranges, the number of dosage events or repeated doses of the analgesic, and the daily total intake levels currently recommended for the treatment of osteoarthritis (OA) and the more severe conditions associated with rheumatoid arthritis (RA). For dosage events, the total dose consumed (typically orally) is not to exceed the maximum value of the daily dosage range in view of safety considerations, although the daily dosage range is also dependent to some extent on the body weight or mass of the patient being treated, and is based roughly on a 100 kg person's weight for the purposes of the present invention. In related embodiments of the present invention, the amounts of collagen and NSAID selected for treatment would be adjusted based on the actual patient's body weight in order to provide the proper dosage or dosage range over the programmed treatment interval for that particular patient or weight range of patients. For women, younger adults and those persons with lower body mass, a proportional amount of actives would be indicated for effective treatment using the compositions of the present invention.

A placebo is included as well for an embodiment that is actually free of or has a dramatically reduced level of one or more analgesics, such as for example, a level at or below the corresponding threshold level of measurable (clinically) therapeutic effect, as it is known in the art that the so called “Placebo Effect” is a psychologically measurable effect whereby a patient who believes they are receiving a medicant, but in fact is not, will still respond positively as if receiving the medicant. More recent studies show, contrary to expectation, that this effect persists, albeit slightly statistically diminished, even if the patient knows that they are taking a placebo, the mere act of taking some corrective action to alleviate a symptom apparently being sufficient to alter the mind's perception to the extent of benefitting from a medicant absent an active ingredient or having a sub-clinically therapeutic amount of a selected active ingredient present.

TABLE 1

| | | | Suggested | Suggested |
|---|---|---|---|---|
| | Daily Dosage | Dosage | for OA | for RA |
| | Range | Events | (Daily, mg | (Daily, mg |
| NSAID (1) | (mg) (2) | (per day) (3) | total) (4) | total) (5) |
| Aspirin | 50-600 | 1-3 | 80 | 600 |
| Celecoxib | 200 | 2 | 100 | 200 |
| Diclofenac | 100-200 | 1 | 100 | 200 |
| Ibuprofen | 300-800 | 3-4 | 1200 | 3,200 (6) |
| Indomethacin | 50-200 | 2-3 | 100 | 200 |
| Naproxen | 500-1000 | 1-2 | 500-1,000 | 500-1,000 |
| Oxaprozin | 600-1200 | 1 | 1,200 | 1,800 |
| Piroxicam | 20 | 2 | 20 | 20 |
| Placebo (7) | 0-2000 | 1-4 | — | — |

(1) Non-steroidal anti-inflammatory drug
(2) Average over-the-counter (OTC) typical dosage values.
(3) Total daily dosage indicated as delivered using multiple dosage events per day.
(4) Suggested dosage level for osteoarthritis, 100 kg adult, human male.
(5) Suggested dosage level for rheumatoid arthritis.
(6) Physician monitoring or prescription recommended at higher dosages.
(7) Inert material not necessarily indicated or identified to user.

### Synergistic Ratios

The preferred ratios of NSAID:collagen vary over a range for each NSAID, and over a wider range for the set of the selected non-steroidal anti-inflammatory drugs of the present invention, depending on the NSAID and to some extent the degree of inflammation or injury at the site subject to arthritis and musculoskeletal distress degeneration, and the nature of the injury. The variation reflects the injury-response mechanism typical of a mammalian body wherein as a course of treatment effects a reduction in inflammation the symptoms of localized pain, swelling and lymphatic buildup are subsequently mitigated or reduced, the effectiveness of the analgesic generally increasing as the amount present in the blood and tissues results in an increased mode of activity with respect to the original extent of musculoskeletal distress symptoms having being subsequently reduced, e.g. or in other words less medicant is required for a smaller or reduced injury than the original injury where symptoms are generally at a maximum starting point when medicants are first administered.

However, the treatment means of the present invention are also suitably employed in a prophylactic manner in inventive embodiments selected to treat the symptoms of arthritis and musculoskeletal distress degeneration before those symptoms reach an undesirable degree of discomfort or pain. In related embodiments, the initial dosage level of the NSAID may be selected to be near its maximum recommended daily dosage limit for treatment of the selected injury. In further embodiments, a continuing dosage level of the NSAID may then be reduced to a level below the initial starting dosage level for a selected period of time, and optionally reduced to an even lower effective dosage level for another selected period of time, in any number, n, of steps, corresponding to the duration of the treatment period. In one embodiment of the present invention, the level of the NSAID may eventually reach zero over the course or duration of the treatment regime, with the remaining administration of medicine being the collagen component alone, without NSAID present. In yet other embodiments of the present invention, the level of the NSAID may be reduced to a sub-clinical level or zero, so that the remaining course of treatment during the remaining period of the treatment regime acts as a prophylactic or remedial treatment means to provide the patient with a maintenance level of dietary collagen as a reserve against further injury.

When treating the symptoms of arthritis and musculoskeletal distress degeneration according to the methods and formulations of the present invention, the amount of the NSAID may be suitably reduced to some extent over the duration of the time of the treatment regime to reflect the body's decreased need for the analgesic portion of the treatment regime.

In Table 2, the daily dosage ranges of some acceptable NSAIDs are presented along with their corresponding synergistic ratio ranges with respect to the native Type II collagen corresponding to SEQ ID NO. 1, according to one embodiment of the present invention.

TABLE 2

| | | Synergistic |
|---|---|---|
| | Daily Dosage | Ratio Range |
| | Range | (Low-High) |
| NSAID (1) | (mg) (2) | (mg) (3) |
| Aspirin | 50-600 | 200-6,000 |
| Celecoxib | 200 | 800-2,000 |
| Diclofenac | 100-200 | 400-2,000 |
| Ibuprofen | 300-800 | 1,200-8,000 |
| Indomethacin | 50-200 | 200-2,000 |
| Naproxen | 500-1000 | 2,000-10,000 |
| Oxaprozin | 600-1200 | 2,400-12,000 |
| Piroxicam | 20 | 80-200 |
| Collagen (4) | 0.10-0.50 | 80-24,000 (5) |

(1) Non-steroidal anti-inflammatory drug.
(2) Average over-the-counter (OTC) typical dosage values.
(3) Synergistic ratio range for selected collagen dosage per 100 kg body mass.
(4) Here, native Type II collagen corresponding to SEQ ID NO. 1.
(5) Lowest and highest of synergistic range for all NSAIDs in this Table.

### Dosage Regimens

Embodiments of the compositions and methods of the present invention produced using the NSAID and collagen may take a wide variety of forms, such as pills, tablets, caplets, capsules, lozenges and the like that are preferably suitable for oral administration and, in this case, are especially preferred to be in unit dosage form although bulk forms such as liquids, syrups, suspensions or linctuses may also be employed. Where the medicament is in unit dosage form it may, for example, be in the form of a tablet or filled capsule (filled with a liquid fill or a particulate or solid fill). The unit dosage form may be formulated to give immediate release of the active ingredients upon administration or may be adapted to give delayed or sustained release or, in indeed, a combination of both immediate and delayed or sustained release of either one or both of the NSAID, collagen and other optional ingredients present.

Sustained release formulation may be produced by encapsulating or spheronizing the active ingredient(s) with either a coating material or a spheronizing agent, respectively. Further, the active ingredients may be melt pelletized in conjunction with a hydrophobic fusible carrier, for example hydrogenated castor oil, hydrogenated vegetable oil, beeswax or carnauba wax, or the like. If desired, a dissolution release control agent may be employed together with the fusible carriers and examples of such include water-soluble organic materials such as pol alkylene glycols or powdered solids such as dicalcium phosphate. The content of NSAID, collagen and any optional opioid analgesic in any particular dosage form will depend upon a number of variables including the number of doses intended to be administered per day and the intended daily dosage desired of the individual actives.

In one embodiment, the effective dose of the collagen material is fixed at a certain, preselected and constant value as determined by the injury or symptoms to be treated, and/or selected by the patient, and the effective dose of the NSAID analgesic is also fixed at a certain, preselected and constant value as determined by the injury or symptoms to be treated, so that the two materials are delivered at a fixed and continuingly constant level over a predetermined treatment time or regime.

In another embodiment, the effective dose of the collagen material is fixed at a certain, preselected and constant value as determined by the injury or symptoms to be treated, and/or selected by the patient, while the effective dose of the NSAID analgesic is varied over the treatment period. In other related embodiments of the present invention, the NSAID dosage is started at a first, higher active level and subsequent doses are reduced in activity, either continuously or step-wise, to a second, lower active level or zero-active level over the predetermined treatment time or regime.

In the various embodiments of the present invention as disclosed herein, the ratio of NSAID:collagen generally starts at the higher end of the synergistic ratio range at the start of, and during the first one or two steps or weeks in the treatment regime. In related embodiments, the ratio of the NSAID:collagen then is reduced at an intermediate step in the treatment regime toward an intermediate synergistic ratio or range, and then is further reduced at a more advanced or the remaining final steps in the treatment regime, to reflect the decreased need for NSAID as the symptoms of the musculoskeletal distress are reduced along with a reduction in perceived discomfort or pain by the patient over the duration of the treatment regime, as well as the improved effectiveness of the NSAID as the healing process reduces the need for the analgesic component of the compositions of the present invention.

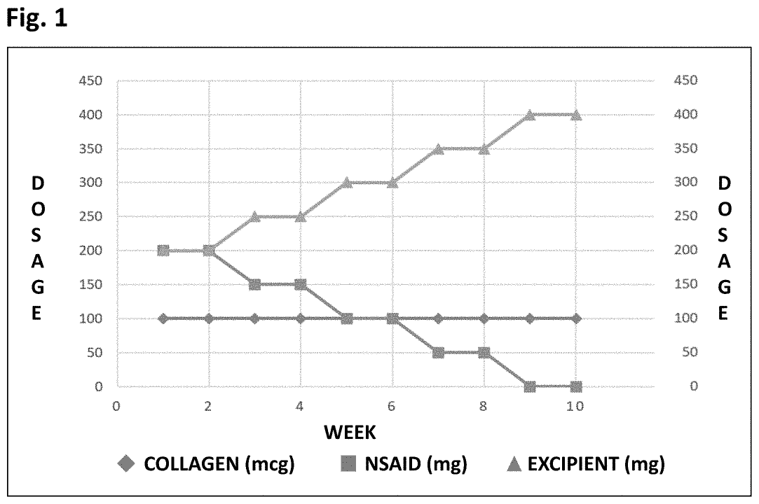

In one embodiment of the present invention, a ten (10) week dosage regime provides a convenient treatment program for a person suffering the effects of musculoskeletal distress by providing a daily regimen consisting of a single pill taken once per day that doses the patient with a synergistic ratio of collagen and an NSAID, here the example being ibuprofen as shown in FIG. 1. For the first two weeks of treatments (14 dosages), the level of NSAID provided is 200 mg and the level of collagen is 100 mcg (0.10 mg), the latter level remaining constant for each dosage event (pill) throughout the duration of the treatment period of ten weeks. For the next two week segment, the level of ibuprofen is reduced to 150 mg, still providing a synergy of action by the combined influence of the NSAID and the collagen in reducing inflammation and aiding in the recovery of the injury being treated. In the third two week segment, the level of ibuprofen providing is reduced yet again to 100 mg, and then in a fourth two week period immediately thereafter the level of ibuprofen is reduced further to 50 mg dosage, so that during this fourth two week segment a combined dosage of 50 mg ibuprofen and 100 mcg of collagen are being provided to the patient in a single convenient pill taken once per day. Finally, during the last or remaining two week segment of the treatment period, the level of NSAID is reduced to zero (absent), providing the patient with only the active level of collagen as initially and continuously present during the dosage treatment period. This approach acts to prevent habituation of the body to the higher dosages of the NSAID, helping to preserve the effectiveness of these drugs to treat additional or ongoing injuries, and to maximize the effectiveness of the present inventive approach of treating these types of diseases and injuries using synergistic combinations of NSAID and collagen without developing habituation or reduced effectiveness owing to continuous exposure or over-exposure of the body's immune system to the effects of the NSAID. Also shown in FIG. 1 is the level of excipient, being the balance of the mass of the dosage form, here for example being a compressible starch whose weight is selected to vary from 200 to 400 mg in steps corresponding to the biweekly dosage segments, as a filler and tableting aid to maintain the dosage form in pill form and provide a uniform weight for the pill form illustrated here.

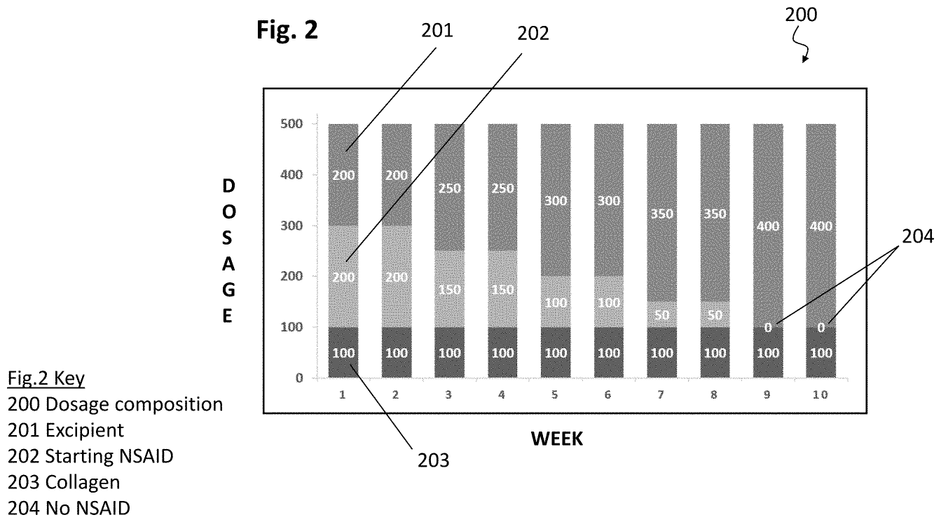

FIG. 2 helps to illustrate the above embodiment of the present invention in additional detail, showing the dosage composition 200 f each series of pills in each weekly segment of the 10 week treatment period presented, where the excipient level 201 starts at 200 mg denoted by the upper section of the bar graph, the NSAID (ibuprofen her) level 202 starts at 200 mg, denoted by the middle section of the bar graph, and the collagen level 203 starts and is maintained at 100 mcg (note the mixed units, so that the graph is not necessarily to scale) throughout the duration of the treatment cycle for each weekly segment up to 10 weeks. Finally, in the final two week segment of the treatment cycle, the level of ibuprofen 204 is reduced to zero, so that the pills provided during this segment are composed only of the excipient (filler here) and the collagen component.

Table 3 shows multiple embodiments of an exemplary dosage regime having weekly steps of a constant, fixed compositional dosage form, varying weekly, biweekly or triweekly, over the duration of the entire treatment period, here illustrated as a 12 week treatment period for Example No. 1, 3-9. In one embodiment of the present invention, shown as Example No. 2, a combination of aspirin and collagen suitable for a shorter four week treatment period is presented.

TABLE 3

| | Daily Dosage | Week | | | | | | | | | | | |
|---|---|---|---|---|---|---|---|---|---|---|---|---|---|
| NSAID (1) | Max (mg) (2) | 1 | 2 | 3 | 4 | 5 | 6 | 7 | 8 | 9 | 10 | 11 | 12 |
| Aspirin | 160 | 160 | 160 | 160 | 80 | 80 | 80 | 40 | 40 | 40 | 20 | 20 | 0 |
| Celecoxib | 200 | 200 | 200 | 175 | 175 | 150 | 150 | 125 | 125 | 100 | 75 | 50 | 0 |
| Diclofenac | 150 | 150 | 150 | 100 | 100 | 75 | 75 | 50 | 50 | 25 | 25 | 0 | 0 |
| Ibuprofen | 400 | 400 | 400 | 400 | 300 | 300 | 300 | 200 | 200 | 100 | 100 | 50 | 0 |
| Indomethacin | 100 | 100 | 75 | 75 | 50 | 50 | 50 | 25 | 25 | 25 | 25 | 25 | 0 |
| Naproxen | 500 | 500 | 500 | 400 | 400 | 300 | 300 | 200 | 200 | 100 | 100 | 50 | 0 |
| Oxaprozin | 800 | 800 | 700 | 600 | 600 | 500 | 500 | 400 | 400 | 300 | 200 | 100 | 0 |
| Piroxicam | 20 | 20 | 20 | 20 | 15 | 15 | 15 | 10 | 10 | 10 | 5 | 5 | 0 |
| Collagen (3) | 0.10 | 0.10 | 0.10 | 0.10 | 0.10 | 0.10 | 0.10 | 0.10 | 0.10 | 0.10 | 0.10 | 0.10 | 0.10 |

(1) NSAID may optionally be enterically coated
(2) Starting or maximum recommended daily dosage
(3) Collagen level per 100 kg subject weight

In other related embodiments of the present invention, the treatment period can be as short a one week, or as long as one year, or alternative can be any intermediate period of duration over which the NSAID can be varied from an initially higher starting dosage to a final reduced or zero dosage at the termination of the treatment period, or at an intermediate point prior to the termination of the treatment period.

Accordingly, in one embodiment involving a treatment period of one month duration, the level and ratio of NSAID:collagen would be varied to start at an initial level at the commencement of the treatment period, decreasing stepwise during the course of the treatment period and terminating with a final dosage consisting essentially of only the collagen component of the dosage form, after which the patient could start an addition, identical treatment series of the same treatment period of one month duration, repeated monthly each month for a total of twelve months or one year, optionally repeated ad infinitum as needed by that particular patient.

In related embodiments, the level of NSAID, collagen and/or the NSAID:collagen level in one treatment regime can be different that the levels of these materials in a second treatment regime, to reflect treatment of a more severe injury or damage for example by a first treatment regime followed by said second treatment regime with a lower dosage or maintenance dose of one or more of said active ingredients tailored to the individual patient to enable more control over the dosage, yet provided in the form of a convenient treatment regime or dosage regimen.

In further related embodiments of the present invention, treatment using the compositions and methods as disclosed herein, may be in the form of a shorter treatment period of weeks or months duration, in which the treatment period is repeated by the individual patient. A key advantage of using the compositions and methods of the present invention include the diminished dependence and development of resistance to or habituation towards, the NSAID component, so that the NSAID can produce a more consistent and beneficial effect over the duration of the treatment period compared to its being orally consumed at a constant level throughout that period.

### Opioids

In other embodiments of the present invention, a stronger analgesic may be desired in order to control the symptoms of pain, including the additional formulation and use of opioid drugs either to replace the NSAID agents or used in combination therewith. Examples of opioid analgesics which may be used in accordance with the invention include, but are not limited to, morphine, hydromorphone, codeine dextropropoxyphene, oxycodone, hydrocodone, and dihydrocodeine, and their pharmaceutically acceptable salts. Generally, when opioids are included, the analgesically effective level of opioid used is desirably lower than the analgesically effective level of the non-steroidal anti-inflammatory drug also present, to reduce a particular patient's exposure to the stronger opioid analgesics, which are classified as narcotics, and likely require prescription by a licensed physician or pharmacologist, and monitoring of the patient to prevent abuse, overdose or habituation.

When included, the typical dosage range of an opioid analgesic is between 10 milligrams to about 1.0 gram, or alternatively between 25 milligrams to 500 milligrams, or alternatively between 50 milligrams to 250 milligrams, and may optionally be adjusted for the weight of the particular patient so that the dosage is dependent on the body mass to prevent overdosing or habituation of the drug.

When included, the dosage of an opioid analgesic is preferably reduced in proportion to the amount of NSAID present, reflecting the lower level of total analgesic needed in a particular treatment step or cycle over a treatment regime of a selected duration of time.

### Additional Ingredients

In addition to excipients and fillers that may optionally be added to the inventive compositions as discussed hereinbelow, other molecular compounds with at least one health benefit may optionally be combined with one or more embodiments of the present invention, including for example, but not limited to, nutritionally required metals such as iron, calcium and magnesium, as well as metal chelates, compounds that can be chelated with metal ions to provide some additional anti-inflammatory properties. Other active ingredients that may be added to the inventive compositions include the following: (1) curcuminoids, for example, but not limited to, those materials and derivatives isolated from turmeric and curcumin species of plants; (2) flavoninols, for instance those extracted from tomatoes containing the maize transcription factor genes Lc and C1; (3) antioxidants, for example those metabolites found in tomato fruit constitutively expressing the grapevine stilbene synthase gene and others known in the art such as Vitamin C and Vitamin E; (4) anthocyanins; (5) caffeoylquinic acid; (6) phenylpropanoid, and related common compounds found in tomato fruit; (7) quercetin; and (8) amino acids, for example, but not limited to glutamine and the other set of 21 naturally occurring amino acids and their isomers.

When present, these additional ingredients may be included in the inventive compositions at levels of between 1 mg to 10 g, or alternatively between 5 mg to 5 g, or yet alternatively between 10 mg to 1 g.

### Excipients

Excipients are a class of materials, being a substance formulated alongside the active ingredient(s) of a medication, included for the purpose of long-term stabilization, bulking up solid formulations that contain potent actives, and to enhance the delivery and/or uptake of one or more active ingredients. Suitable excipients for the instant invention include, but are not limited to, antiadherents, binders, coatings, colorants, disintegrants, dispersants, encapsulants, flavors, glidants, lubricants, preservatives, sorbents, sweeteners, vehicles, and combinations thereof. An excipients is an optional ingredient, and when employed, may constitute a small percentage by weight or volume, or a larger percentage by weight or volume, of the final formulated product. When present, an excipient may be present at a level or weight sufficient to achieve its effect, constituting the remaining volume or weight of the dosage form in order to produce a uniformly weighted series of dosages within a treatment regime, for ease of handling, packaging and subsequent oral intake by a person undergoing the treatment according to one or more embodiments of the present invention.

### Fillers

Fillers are a class of inert, non-active materials, being a substance formulated alongside the active ingredient(s) and/or excipients of a medication, included for the purpose of bulking, tablet or capsule formation, so that the medication can be produced having a certain size, structure or configuration regardless of the actual amount of the analgesic components and collagen present. A filler is an optional ingredient, and can include colorants and compression aids that ease tablet or capsule formation and provide a distinctive appearance to the final formulated product for quality control and identity purposes. When employed, the filler may constitute a small percentage by weight or volume, or a larger percentage by weight or volume, of the final formulated product. In one or more of the embodiments of the present invention as disclosed, a filler is employed as a bulking material constituting the remaining volume or weight of the dosage form, with respect to the total combined weight of the one or more pharmaceutically active drugs used, in order to produce a uniformly weighted series of dosages within a treatment regime, for ease of handling, packaging and subsequent oral intake by a person undergoing the treatment according to one or more embodiments of the present invention.

### Dosage Means

Dosage means for providing the compositions of the present invention and for ease of use in following the methodology of a programmed dosage regime wherein the levels of NSAID vary while still providing a synergistic ratio of NSAID:collagen, include any suitable formulation and packaging means know in the art. For example, compositions of the present invention can be formulated by simple mixture of the active components and excipients and/or fillers in the form of pills, tablets, caplets, lozenges and corresponding liquid, serum and gel formulations that can physically and chemically stabilize the active materials until the time of use or oral ingestion, and optionally also provide a standard or uniform dosing regimen providing convenience to the user, so that they take a single pill or multiple of a single pill per day, without needing to account for variations in the composition of the dosage of the individual ingredients that may change per treatment segment or cycle.

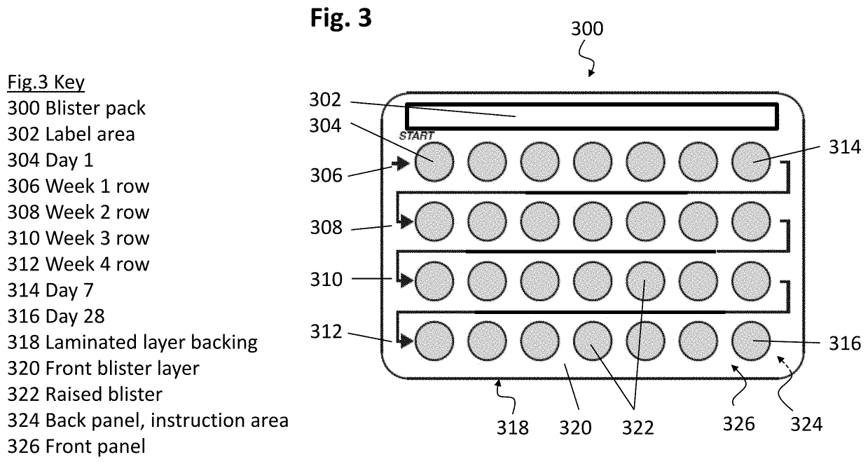

In one example embodiment shown in FIG. 3, a blister pack 300 provides the inventive compositions needed for a one month treatment segment or treatment cycle (if repeated monthly or extended monthly), the monthly segment or cycle identified to the user in the label area 302 so that the correct sequence of pills provided by the blister pack 300 can be taken, orally, once daily in this embodiment. The individual pills are removed from the blister pack 300 by pressing on the front side 320 of the individually encapsulated pill in a raised blister 322 located on the front panel 326 of 300, in order to break the rear foil seal 318 (not shown) on the back side or back panel 324 (not shown) in the immediate location of the pill, for example, the first pill 304, to release just that first pill 304 and then subsequent pills one at a time by pressing down to release them individually as needed. Here, the first week series of pills is provided in a first row 306 of which pill 304 is the Day 1 pill, and pill 314 is the Day 7 pill. Following the first week segment, pill 308 is Day 8 or the first pill of the second week segment. The pills are then taken in order for each weekly segment, the next weekly segment 310 and the final weekly segment 312 until all the pills have been consumed by the patient, following a monthly treatment cycle. Instructions for use can be printed for convenience on the rear panel 324, as well as in the label area 302 located on the front panel 326 as shown in FIG. 3.

In other embodiments, other configurations of blister packs can be suitably employed, or other means such as the use of dispensers, bottles, and other packaging means to sort and present the dosage forms of the present invention in the correct order for being taken by the patient following the treatment regime.

In further embodiments, other means such as the use of shapes, colors and/or designs formed or imprinted onto the pills, caplets, tablets, capsules and the like can be employed to communicate to the user the correct dosage form to take at on any given day, or during any given treatment segment, in order to start and complete the desired treatment cycle of dosages of the inventive compositions as disclosed herein.

The above illustration provides many different embodiments or embodiments for implementing different features of the invention. Specific embodiments of components and processes are described to help clarify the invention. These are, of course, merely embodiments and are not intended to limit the invention from that described in the claims.

Although the invention is illustrated and described herein as embodied in one or more specific examples, it is nevertheless not intended to be limited to the details shown, since various modifications and structural changes may be made therein without departing from the spirit of the invention and within the scope and range of equivalents of the claims. Accordingly, it is appropriate that the disclosure and examples presented be construed broadly and in a manner consistent with the scope of the invention, as set forth herein.

What is claimed is:

1. A method to treat and alleviate symptoms of musculoskeletal inflammation by administering a dietary oral dosage regimen comprising:
a. a therapeutic amount of an undenatured collagen;
b. a therapeutic amount of an analgesic compound comprising a non-steroidal anti-inflammatory drug selected from aspirin, ibuprofen, and combinations thereof;
c. optionally, a pharmaceutically acceptable excipient;
wherein said dietary dosage regimen comprises a treatment period whose duration is selected from 1 week to 1 year.

2. The method of claim 1 wherein said undenatured collagen is a Type II collagen.

3. The method of claim 2 wherein said undenatured Type II collagen is derived from an avian source.

4. The method of claim 3 wherein said avian source is chicken and cartilaginous components thereof.

5. The method of claim 1 wherein said therapeutic amount of said analgesic compound varies over said treatment period; wherein said amount of analgesic is provided at a first dosage level during a first series of administrations; followed by a second dosage level over a second series of administrations; and optionally followed by an n-plurality of additional dosage levels over said plurality of series of continuing administrations;
wherein said dosage regimen delivers a total of n+2 administration series over said duration of said treatment period.

6. The method of claim 1 wherein said undenatured Type II collagen corresponds to an expressed cross-linked polypeptide coded by the nucleic acid molecule consisting of a sequence of bases encoding the amino acid sequence of Seq. ID. No. 1.

7. The method of claim 1 wherein each of said second and n-plurality of additional dosage levels deliver dosage levels of said analgesic at a lower level than said first dosage level.

8. The method of claim 7 wherein each of said n-plurality of additional dosage levels deliver dosage levels of said analgesic at progressively lower levels than said first and said second dosage levels.

9. The method of claim 8 wherein said final n+2 dosage levels of said analgesic compound corresponds to a subtherapeutic dosage level of said analgesic.

10. The method of claim 1, wherein said total of administration series corresponds to an oral dosage regiment extending over a time period selected from 2, 4, 6, 8 and 12 weeks.

11. A method for the inhibition of an arthritis disease process in a patient in need thereof, the method comprising: orally administering a non-steroidal anti-inflammatory drug (NSAID) or pharmaceutically acceptable salts thereof; and
an undenatured Type II collagen in synergistic amounts and in a ratio of NSAID:collagen from about 80:1 to about 12,000:1; and for a duration effective to inhibit an arthritis disease process in a patient in need thereof.

12. The method according to claim 11, wherein said non-steroidal anti-inflammatory drug (NSAID) is selected from aspirin, aspirin salsalate, celecoxib, diclofenac, diflunisal, etodolac, ibuprofen, indomethacin, ketoprofen, ketorolac, nabumetone, naproxen, oxaprozin, piroxicam, salsalate, sulindac, and tolmetin.

13. The method according to claim 11, wherein at least one of said NSAIDs are in an oral dosage form suitable for dosing once or twice a day over a treatment period.

14. The method according to claim 11 wherein said NSAID is administered in a daily dose ranging from about 100 to about 2,400 milligrams and said collagen is administered in a daily dose ranging from about 50 to about 500 micrograms.

15. The method of claim 11, wherein said arthritis disease is selected from osteoarthritis, rheumatoid arthritis or osteopenia, or combinations thereof.

16. The method of claim 11, wherein said collagen is an undenatured Type II collagen.

17. The method of claim 16, wherein said undenatured Type II collagen corresponds to an expressed cross-linked polypeptide coded by the nucleic acid molecule consisting of a sequence of bases encoding the amino acid sequence of Seq. ID. No. 1.

18. The method of claim 13, said oral dosage form comprising a variable amount of 5 to 250 milligrams of a NSAID administered over said treatment period and a fixed amount of 50 to 500 micrograms of an undenatured collagen administered over said treatment period.

19. The method of claim 18, wherein said dosage form comprises a first dosage level during a first series of administrations; followed by a second dosage level over a second series of administrations; and optionally followed by an n-plurality of additional dosage levels over said plurality of series of continuing administrations; wherein said dosage regimen delivers a total of n+2 administration series over said duration of said treatment period.

20. The method of claim 19, wherein said NSAID and said collagen are present in synergistic amounts over the duration of said treatment period and present in a synergistic ratio of NSAID:collagen from about 80:1 to about 12,000:1;
wherein said synergistic ratio varies over said treatment period; and wherein said treatment period is of a duration effective to inhibit an arthritis disease process in a patient in need thereof.
